(19) United States
(12) Patent Application Publication
(10) Pub. No.: US 2014/0288558 A1
(43) Pub. Date: Sep. 25, 2014

(54) Flexible Rail Bar Bone Fixation System

(71) Applicant: Javier Montejo, Neiva (CO)

(72) Inventor: Javier Montejo, Neiva (CO)

(21) Appl. No.: 13/846,980

(22) Filed: Mar. 19, 2013

(51) Int. Cl. A61B 17/64; A61B 17/86

(52) U.S. Cl. CPC A61B 17/6433; A61B 17/864
USPC 606/56; 606/304

(57) ABSTRACT

Flexible Rail Bar Bone Fixation System is a type of intermaxillary fixation (IMF) system consisting of an inner rail bar which fixes to the jaw through the use of bone anchorage screws. These screws have an elongated internal threaded nut head with the function of receiving a second screw to secure the rail bar. The elongated head allows for the separation of oral tissue from the rail bar.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

1. Field of the Invention

The present invention relates, in general, to the fixation of bone fractures, and more particularly to the jaw for holding any possible fractured bone fragments.

2. Description of Related Art

Within the field of oral and maxillofacial surgery one of the most important phases in the handling of fractures is intermaxillary fixation (IMF), due to the fact that during this step the patient's original occlusion is reestablished.

Historically, intermaxillary fixation systems have been based on the use of teeth as anchorage for interdental surgical wires such as Erich, Ivy or Kazanjian arch bars, which are used in procedures that require maxillary fracture reductions. Notwithstanding their functionality, these systems have a number of disadvantages for both patient and surgeon, being the health of the gums the main issue at hand after their prolonged use.

For the patient, these systems cause discomforts and ailments in cases when the injuries are reversible, and when irreversible, they can produce grave periodontal disease. For the surgeon, these fixation systems require the existence of intact teeth in order to do the procedure, a situation that is often difficult when faced with severe fractures. Additionally, their troublesome wire-based installation represents a biological hazard.

In both cases, the use of a less invasive system can avoid these inconveniences.

During the last few years a solution to these problems has been presented with the appearance of IMF bone screws that have allowed a rapid intermaxillary fixation. This system however has other technical issues, e.g. it doesn't provide sufficient stability to the fixation, producing posterior open bites, and their difficult installation in complex traumas.

Improvements to the customary approach to fracture fixation have been proposed and are disclosed and discussed in the following exemplary patents.

The foregoing patents and other prior art reflect the current state of the art of which the present inventor is aware. Reference to, and discussion of, these patents is intended to aid in discharging Applicant's acknowledged duty of candor in disclosing information that may be relevant to the examination of claims to the present invention. However, it is respectfully submitted that none of the above-indicated patents disclose, teach, suggest, show, or otherwise render obvious, either singly or when considered in combination, the invention described and claimed herein.

The following patents are references to similar patents:

| | | |
|---|---|---|
| 2,502,902 | April 1950 | Tofflemire |
| 4,797,095 | January 1989 | Armstrong et al. |
| 5,087,202 | February 1992 | Krenkel |
| 5,839,899 | November 1998 | Robinson |
| 5,842,856 | December 1998 | Casey |
| 6,086,365 | July 2000 | Fields |
| 6,227,861 | May 2001 | Cartledge |
| 6,257,884 | July 2001 | Chang |
| 6,595,774 | July 2003 | Risse |
| 7,351,058 | April 2008 | Fore et al. |
| 2001/0018176 | August 2001 | Branemark |
| 2002/0013586 | January 2002 | Justis et al. |
| 2002/0143336 | October 2002 | Hearn |
| 2002/0150856 | October 2002 | Payton |
| 2003/0160552 | August 2003 | Bacho et al. |
| 2004/0086824 | May 2004 | Kesling |
| 2004/0152046 | August 2004 | Minoretti et al. |
| 2005/0059971 | March 2005 | Michelson |
| 2005/0261690 | November 2005 | Binder et al. |
| 2005/0282115 | December 2005 | Gedebou |
| 2006/0069389 | March 2006 | Knopfle |
| 2006/0078849 | April 2006 | Parks |
| 2007/0259306 | November 2007 | Raines et al. |
| 2011/0288596 | November 2011 | Brand et al. |
| 2012/0214120 | February 2012 | Marcus et al. |

### BRIEF SUMMARY OF THE INVENTION

This system is innovative because it does not use teeth as an anchorage point, thereby reducing periodontal disease and improving the stability of the IMF through the use of a tiered bone screw and rail bar. It improves the quality of the surgical procedure for the surgeon and reduces, among other things, gum-related problems for the patient due to the separation of the rail bar from the gum tissue.

Accordingly, an object of the present utility is to provide a flexible rail bar that can wrap around the mandibular region both horizontally and vertically while being secured with a tiered bone screw.

The invention contains two parts—the rail bar and the tiered bone screw. The rail bar is made of flexible metal that can be easily cut to any length that includes at least one enclosed hole for inserting at least two screws. The tiered bone screw is made of metal and is separated in two parts: the bone anchorage screw, which contains an external threading for inserting into the bone and an elongated internal threaded nut head that allows for the separation of oral tissue from the rail bar and the fixation screw which passes through the rail bar and is tightened into the elongated head of the bone screw.

Once the bone anchorage screws have been inserted in their designated locations, the rail bar is laid across the tissue and the fixation screws are inserted into the holes of the rail bar and secured to the bone screws, creating a fixed, rigid rail bar.

After rail bars have been secured to the upper and lower mandibular bone, a smaller connection rail bar with one or two holes is placed over the fixed rail bars and a shaft screw is placed through the holes of both the connection rail bar and the fixed rail bar and secured with a nut that is placed between the tissue and the fixed rail bar.

Rail bars are flexible until the fixation screws are placed, whereas the rail bar becomes fixed in its position.

If necessary, two holes can be combined by cutting off the connection between the two sides of the rail.

Other novel features which are characteristic of the invention, as to organization and method of operation, together with further objects and advantages thereof will be better understood from the following description considered in connection with the accompanying drawings, in which preferred embodiments of the invention are illustrated by way of example. It is to be expressly understood, however, that the drawings are for illustration and description only and are not intended as a definition of the limits of the invention. The various features of novelty that characterize the invention are pointed out with particularity in the claims annexed to and forming part of this disclosure. The invention does not reside in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a schematic side and top view of Bone Anchorage Screw with external and internal threading

FIG. 2 is a schematic side and top view of the Fixation Screw with external threading and Phillips head

FIG. 3 is a schematic side and top view of the Flexible Rail Bar

FIG. 4 is an example of the Tiered Bone Screw in relation to the Flexible Rail Bar.

FIG. 5 is an example of the two parts of the Tiered Bone Screw both separated and together

FIG. 6 is a schematic side view of the Tiered Bone Screw inserted into the bone, through the gum tissue while fixating the Rail Bar. It also shows the connection rail bar attached with a shaft screw and nut.

### DETAILED DESCRIPTION OF THE INVENTION

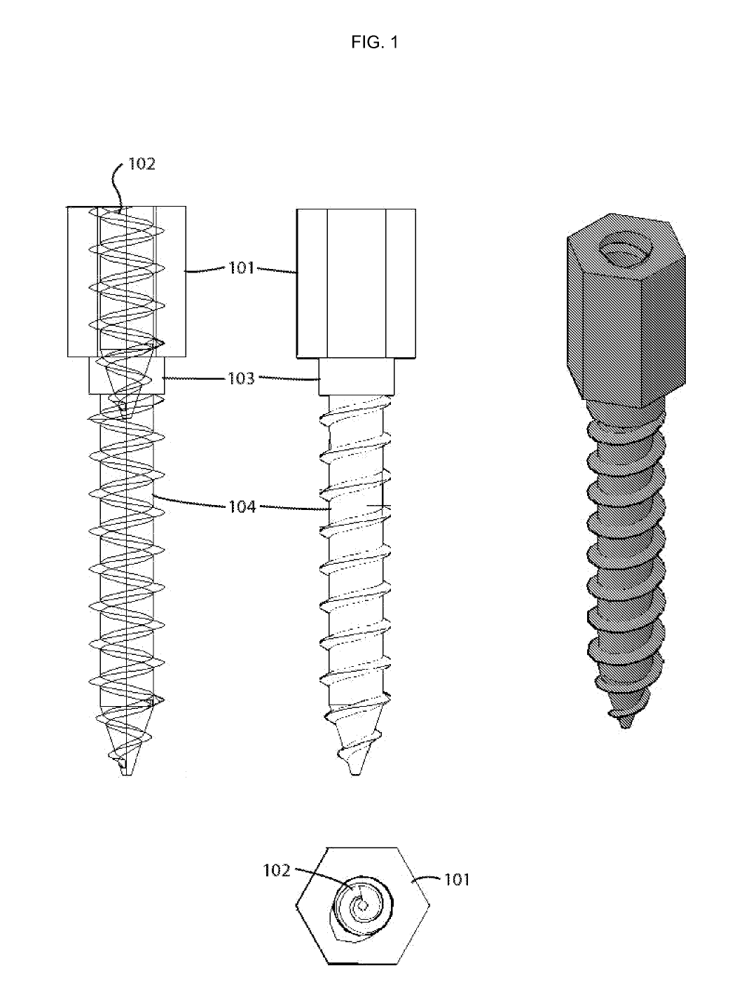

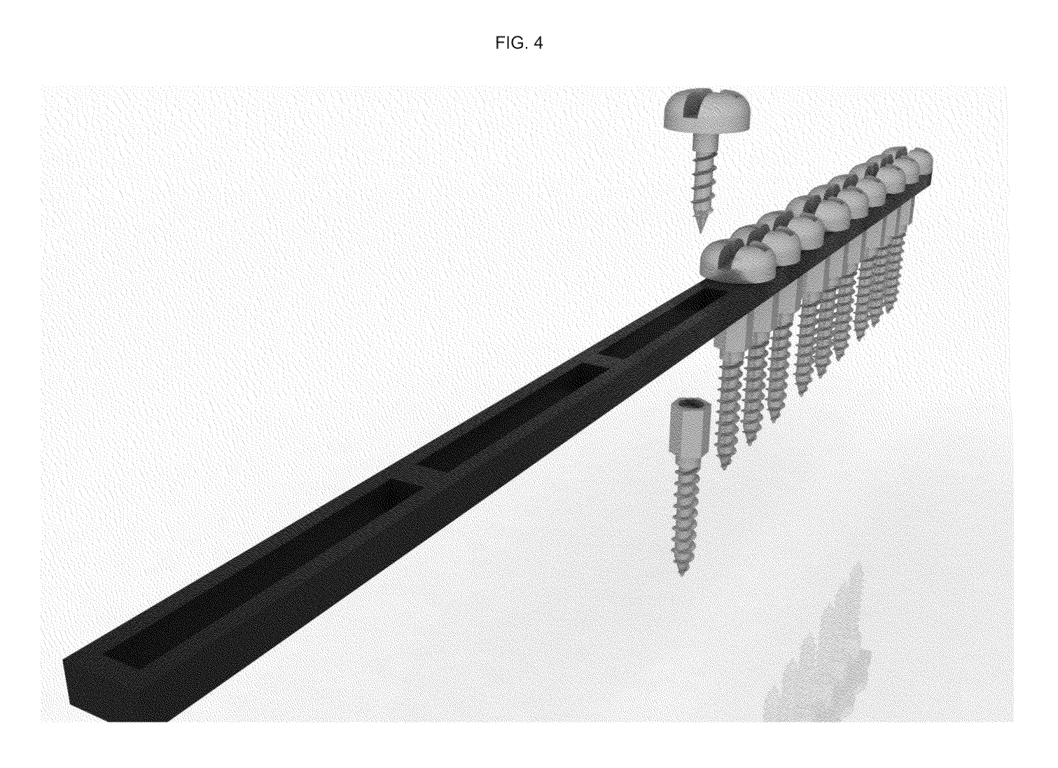

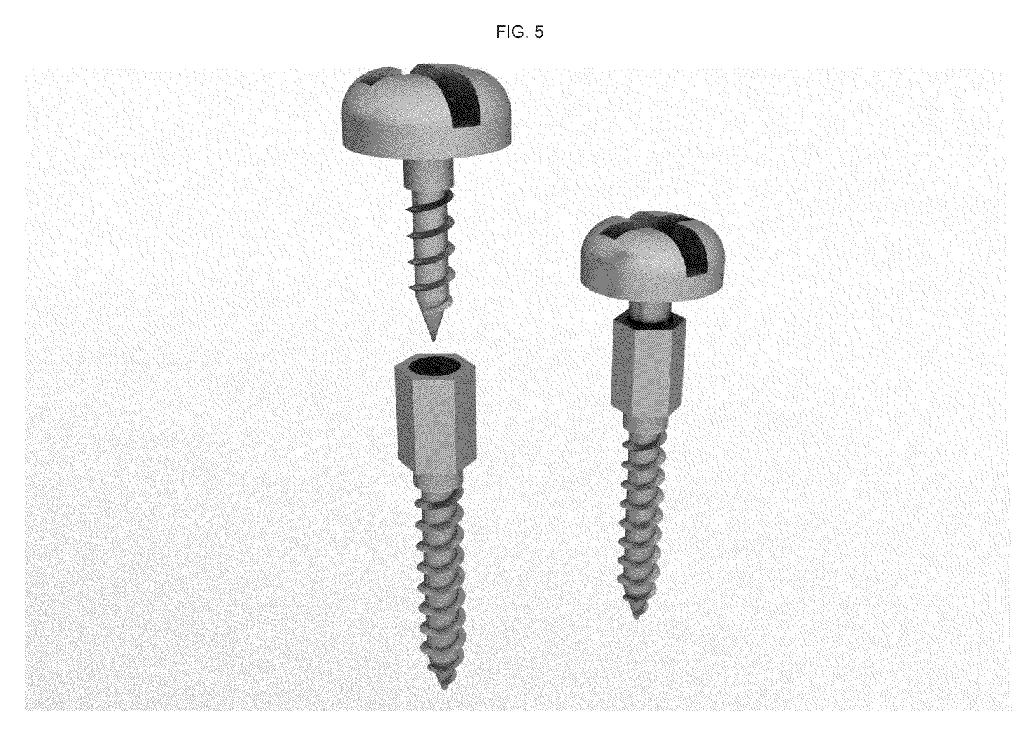

Referring to FIGS. 1 through 5, wherein like reference numerals refer to like components in the various views, there is illustrated therein a Flexible Rail Bar Bone Fixation system with adjustable screw placement depending upon the application. These views show, collectively, an adaptable and easily customized system adapted for interior or exterior use in the fixation of intermaxillary bones.

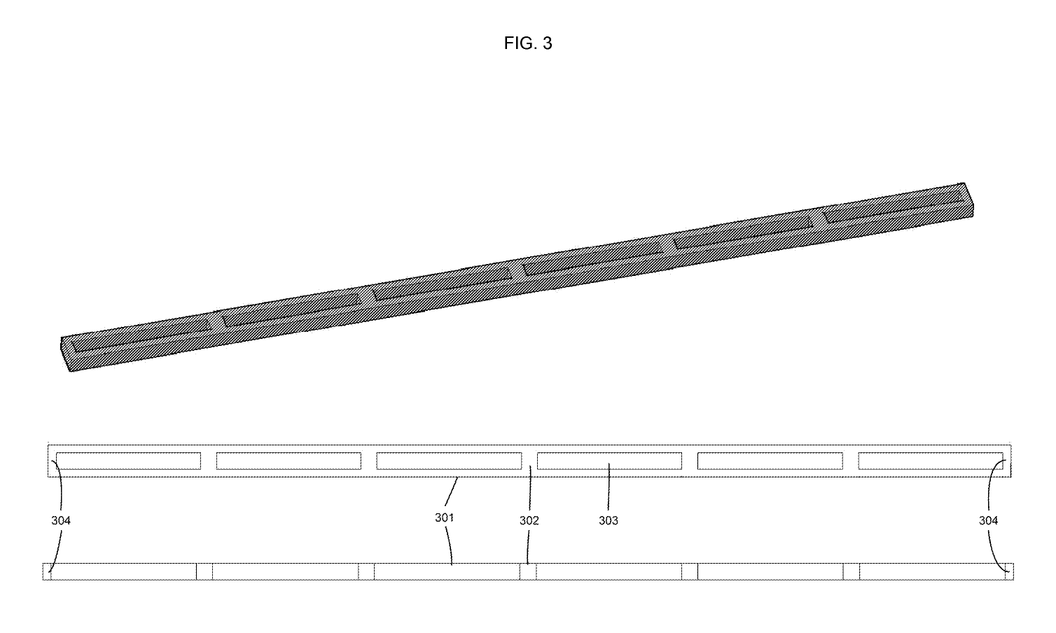

The Bone Anchorage Screw in FIG. 1 is a self-perforating screw that is placed in the designated region of the maxilla (602), taking note not to perforate the dental roots. A minimum of two Bone Anchorage Screws (FIG. 1) must be placed in each maxilla (602) and the elongated hexagonal threaded head (101) will extend out of the oral tissue (601), providing at least 3 mm of gap between the gum (601) and the rail bar (301) shown in FIG. 3.

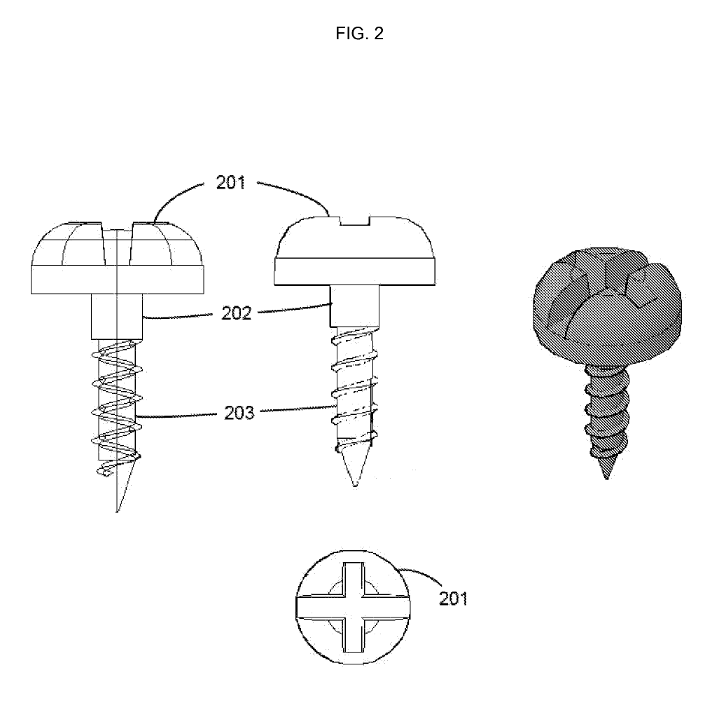

Once the Bone Anchorage Screws (FIG. 1) have been secured, a flexible metallic rail bar (FIG. 3) is placed over the heads of the elongated hexagonal threaded heads (101). The second level of the tiered bone screw (FIG. 5), known as the fixation screw (FIG. 2) is inserted in the holes (303) of the flexible rail bar (FIG. 3) and screwed into the elongated heads (101) of the Bone Anchorage Screws (FIG. 1) until the screws are tight and the rail bar (FIG. 3) is rigid.

This process is then applied to the mandibular bone (602) with the same precautions and procedures.

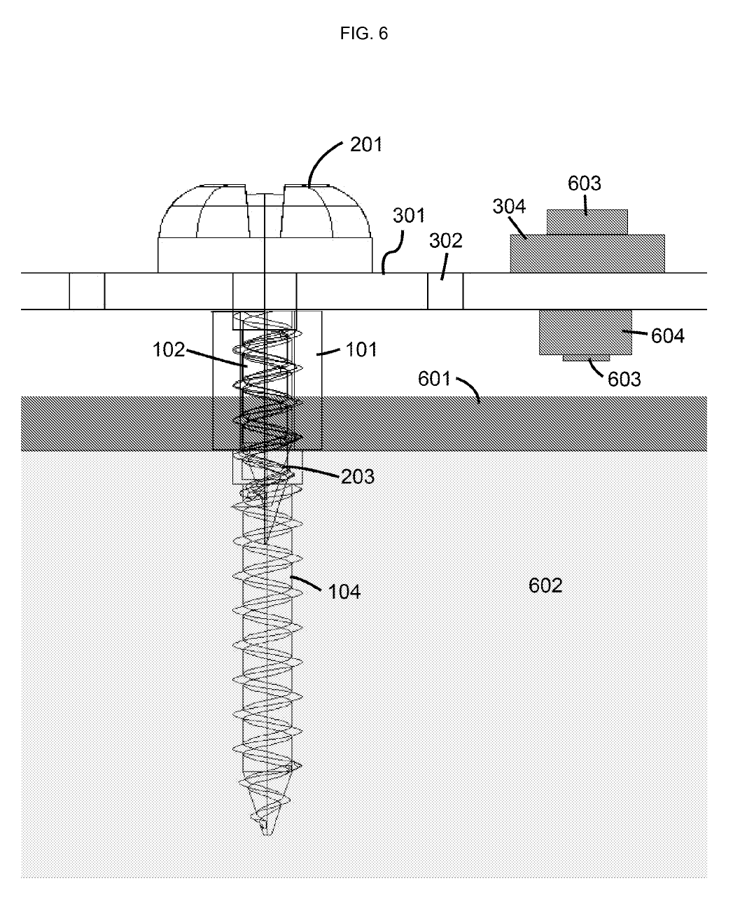

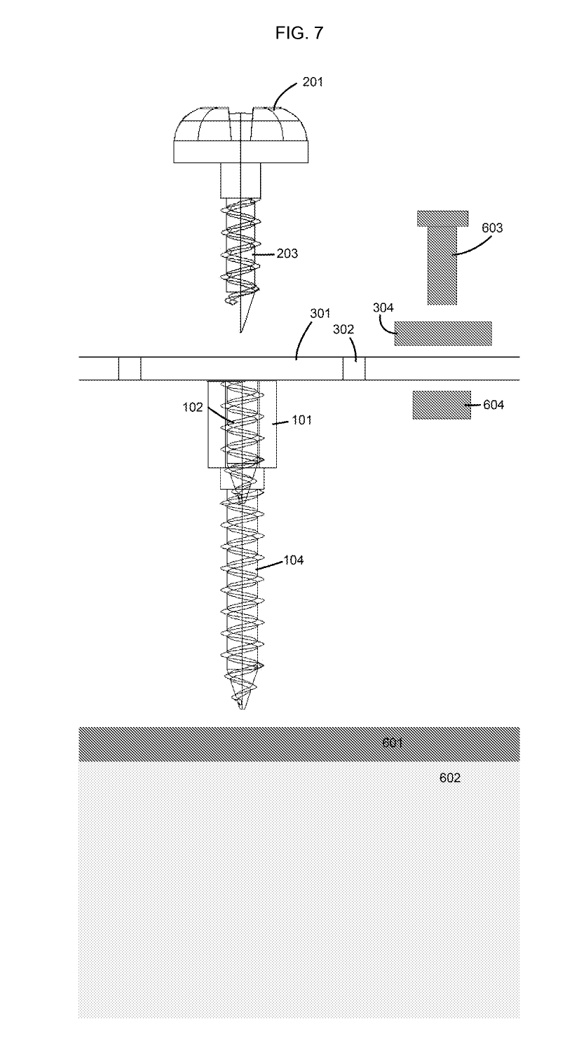

Once both rail bars (FIG. 3) are secured to the bone, a second, smaller rail bar connection (FIGS. 6&7—304) is placed on the intermaxillary achieving the desired dental occlusion. The smaller rail bar (FIGS. 6&7—304) must consist of at least one complete section, which includes two sides (301) and two connections (302). A shaft screw with a flat end (603) is placed through the small rail bar connection (FIGS. 6&7—304) then through the rail bar (FIG. 3) that has been fixed to the bone. The rail bar connection (FIGS. 6&7—304) is adjusted to the desired location and the shaft screw (603) is then secured with a nut (604) that is placed between the gums (601) and the rail bar (FIG. 3).

1. Rail bar characterized by its flexibility and comprised of holes that can be segmented in order to create a rigid point of connection for securing maxilla and mandibular bone fractures.

2. Tiered bone anchorage screw consisting of two levels: a screw with external threading and an elongated hexagonal head with internal threading and a screw that is inserted into the elongated hexagonal head with the sole purpose of securing the rail bar to reduce fractured bones.
